(19) United States
(12) Patent Application Publication
(10) Pub. No.: US 2006/0122542 A1
(43) Pub. Date: Jun. 8, 2006

(54) Wireless physical testing system and method of use

(76) Inventors: Len Smith, Salt Lake, UT (US); Darrell Barnhart, Salt Lake City, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/008,078

(22) Filed: Dec. 8, 2004

(51) Int. Cl. A61B 5/11; A61B 5/00; A61B 5/02

(52) U.S. Cl. 600/595; 128/903; 600/557; 600/549; 600/483; 600/300

(57) ABSTRACT

An improved wireless physical testing system allows for communication between multiple wireless physical testing instruments and a recording device. The capability to use multiple wireless testing instruments simultaneously allows a test operator to perform tests that accurately simulate life activities for tests such as occupational testing, therapy, or disability testing.

### BACKGROUND OF THE INVENTION

1. The Field of the Invention

The present invention relates to a system used in conducting strength and range of motion tests of individual muscles, muscle groups, or joints; in conducting pain and pressure sensitivity tests; and/or in monitoring the heart rate or body temperature alone or in combination with other tests in physical and occupational therapy diagnosis, and individual functional capacity assessment in employment evaluations. More specifically, the present invention relates to an improved wireless testing system which functions in connection with individual testing equipment including but not limited to strength and force testing devices, range of motion testing devices, pain or pressure sensitivity devices, or heart rate monitors and allows tests to be conducted simultaneously with multiple devices, allowing greater flexibility and accuracy in testing.

2. State of the Art

Functional capacity testing and testing of individual muscles, muscle groups, or joints typically involves measuring the range of motion of the indicated joint or the strength of the desired muscle or muscle group, either at specific positions or throughout the range of motion of the joint. Thus, a functional capacity test may measure any of a variety of physical conditions to provide a more accurate picture of a patient's physical state.

In many circumstances it is desirable to measure the heart rate or body temperature before, during, or after testing. Additionally, the sensitivity of a particular body part to pain or pressure is used in assessing injury, recovery, or desired treatment. These types of tests provide valuable information to physical and occupational therapists about the individual's strength or functional capacity at specific joints and muscle groups. These tests also provide information to the therapist about the extent of an injury or disability an individual may have. The tests also allow determination of an individual's ability to function in various capacities in the workplace. Therapists may use these tests to determine the effectiveness of treatment, or to measure an individual's improvement due to the treatment. Sports trainers and other trainers or coaches may use similar tests to measure performance in specific activities and implement strategies to improve the performance in the desired activity. It is desirable for many professionals to measure and or monitor various conditions surrounding a patient or individual's body. These professionals may include but are not limited to chiropractors, neurologists, orthopedic surgeons, and the like.

Muscle strength, range of motion, pain sensitivity, and heart rate tests have historically been performed with hand-held mechanical instruments. Readings from the mechanical instruments are obtained from dials, gauges, or digital displays mounted on the instrument. These mechanical instruments must typically be used while the individual remains generally stationary to allow the person performing the test to take a reading from the instrument. The person performing the test also is typically limited to performing one measurement or test at a time because of the need to read the instrument and record the measurement, although more than one test may be conducted if the testing device includes some sort of memory.

The quality of the test is also limited by the accuracy and repeatability of the instrument and by the ability of the person performing the test to properly use and take measurements from the instrument. Furthermore, when using dials or gauges, it is difficult for the therapist, etc., to record any information other than the absolute value achieved during the test. Thus, for example, the range of motion regarding a limb may only give the absolute range and not indicate points along the range at which the patient hesitated, moved slowly, or otherwise had difficulty moving.

Electronic devices for performing these physical tests have also been used. These devices offer advantages such as increased accuracy, automatic recording of data, increased reliability, more consistent testing, and faster testing. A significant disadvantage of these electronic devices is the wire necessary to connect the device to the power source and measure and record the test results. These wires require that the test be performed within a limited distance, typically a few feet, of the recording device. The wires also clutter the testing area and easily become entangled with other wires or themselves. Further, the wires may prevent a person from fully performing a desired test where the wire's length prevents sufficient movement of the person being tested or otherwise interferes with the testing process. The wires also hinder the use of multiple testing devices as multiple wires can easily become entangled with each other and the individual being tested.

Some electronic testing devices have been made with wireless transmitters to communicate with the recoding device. These devices offer a significant advantage over the wired electronic devices in that the individual being tested is free to move without the restriction of the wire connection. The wireless testing device also reduces the clutter of wires in the testing area and prevents the testing device from becoming entangled with other devices. These devices are currently limited to a few types of testing devices such as pinch strength or simple inclinometry. Thus a person performing tests who desires the accuracy of an electronic device with the convenience of wireless testing is limited to performing a few simple individual tests. Additionally, the person is also limited in using current wireless devices because typically only one device may be used at a time to prevent device interference. Thus a wireless testing device may be limited to use outside the range of other wireless testing devices in use, limiting the use of multiple devices in the same or neighboring offices or testing areas. Additionally, many wired or wireless electronic devices may only record the maximum value achieved during a test and not the range of values generated.

Because of the limitations in current physical testing equipment, a person performing tests is typically limited to performing simple tests that may not accurately represent the activity the test is intended to simulate. A person testing with mechanical equipment is limited to performing single measurements on a stationary subject. Thus, with currently available technology, a person testing with wired electronic equipment is able to perform multiple measurements, but the range of motion of the individual tested is limited by the wires and the individual must be in close proximity to the recording device. The individual tested with wireless electronic testing equipment is able to move freely, but the test may typically be limited to single measurements and limited by other proximate wireless testing equipment.

It is desirable to perform physical tests of greater complexity that more accurately represent and measure the movement or activity of concern. These tests require the greater mobility and freedom of a wireless testing system and require the use of multiple measuring devices. For example, a sports trainer analyzing a baseball pitcher's throw or a golfers swing may desire to measure the range of movement of the athlete and the forces exerted throughout that movement. A person performing disability or functional capacity evaluations may desire to obtain multiple measurements such as heart rate, range of motion, and force exerted while the subject performs occupational or life activities. Such measurements require the use of multiple wireless devices to allow the subject to move about a testing area freely and perform tasks such as lifting or moving objects, climbing, or walking.

It is also desirable for a wireless testing system to be easily transported to different locations for testing. The wireless testing system will preferably communicate to a portable recording device such as a laptop or desktop computer, or a PDA or handheld organizer. This ease of transportation would allow a person to easily take the testing equipment to a home, jobsite, or patient care center for testing to conduct the test while minimizing lost time caused by tangled wires, etc.

There is thus a need to have a wireless physical testing system which allows for simultaneous use of multiple physical testing devices for functional capacity evaluation, disability, and/or other physical testing which allows the operator to perform tests that realistically simulate occupational and life activities.

### SUMMARY OF THE INVENTION

It is an object of the present invention to provide an improved wireless physical testing system and method of use.

One aspect of the present invention is a wireless testing system where the data recording device can identify the wireless testing devices with which it is communicating and not accept signals from other wireless testing devices. Each testing instrument could have an identifying characteristic and the data recording device could accept signals only from instruments in the testing group with which it is associated, or instruments designated for the test being performed.

Additionally, another aspect of the present invention is a data recording device which can automatically detect which testing instruments are in use. The data recording device could monitor for signals sent from the testing instruments and if a testing instrument is used and sends a signal, the data recording device could automatically begin recording the data sent from the instrument and display the data.

Another aspect of the present invention is a wireless physical testing system which is capable of providing a real time display of the measurements from each testing device. According to this aspect of the invention, a testing system could receive data from the testing instruments at a high sampling rate and display the data on a monitor or other display device as it is received, providing a substantially continuous reading of the measured data to the test operator.

Yet another aspect of the present invention is a wireless physical testing system where the individual wireless testing devices can communicate to a recording device, to a laptop or desktop computer, or to a PDA or handheld organizer that is equipped with a proper radio frequency communication device. A testing system capable of communication with a portable recording device could be used at a workplace, hospital, home, or other location.

The above aspects of the present invention are intended as exemplary of the invention and not as limitations of the claims.

### BRIEF DESCRIPTION OF THE DRAWINGS

Various embodiments of the present invention are shown and described in reference to the drawings. It will be appreciated that the preferred embodiments are illustrative but not limiting to the scope of the present invention which is set forth in the appended claims.

FIG. 1 shows a wireless testing system according to an embodiment of the present invention.

FIG. 2 shows a schematic block diagram of an embodiment of the data recording device.

FIG. 3 shows a schematic block diagram of functions that may be performed by the CPU of the data recording device.

FIG. 4 shows a schematic block diagram of a wireless physical testing instrument.

FIG. 5 shows a schematic block diagram of functions that may be performed by the CPU of the wireless physical testing instrument.

FIG. 6 shows a schematic block diagram of an adapter to adapt a wired physical testing instrument to wireless use.

FIG. 7 shows a schematic block diagram of functions that may be performed by the CPU of the adapter to adapt a wired physical testing instrument to wireless use.

FIG. 8 shows a schematic block diagram of an adapter to adapt a portable device to communicate wirelessly with various testing instruments.

### DETAILED DESCRIPTION

The drawings will now be discussed in reference to numerals provided therefore so as to enable one skilled in the art to make and use the present invention. It is to be understood that the drawings and description thereof are for explanatory purposes and are not intended to narrow the scope of the appended claims.

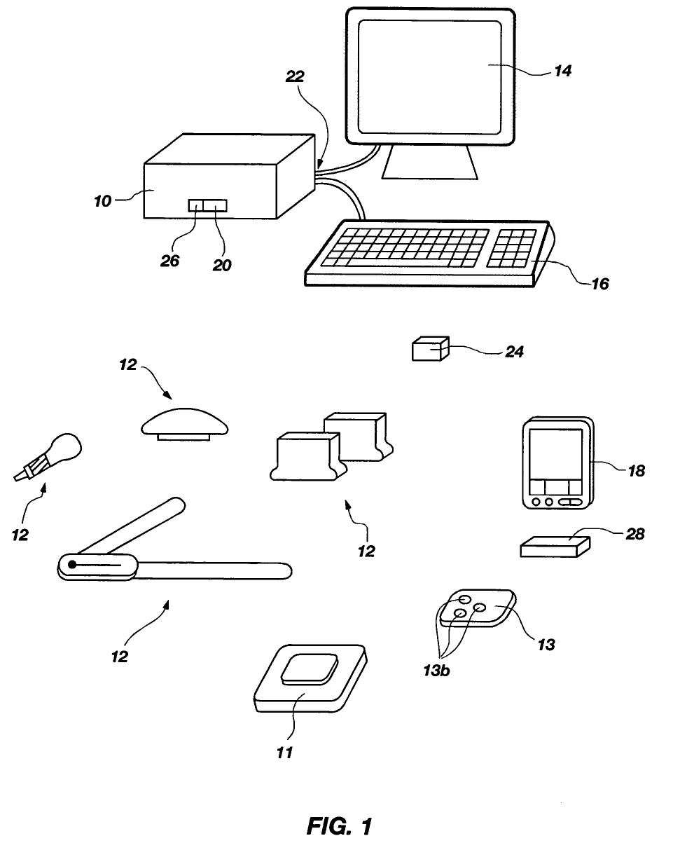

Turning now to FIG. 1, an example wireless physical testing system is shown. The physical testing system includes a data recording device 10. The data recording device 10 communicates wirelessly with various physical testing instruments, indicated generally at 12, such as an algometer, pinch or grip strength testing devices, force testing devices, a goniometer, inclinometers, etc. Accordingly, the data recording device 10 typically would include wireless transmitters and receivers or a transceiver 20 to allow communication with the testing instruments 12 or the portable device 18. It will be appreciated by one of skill in the art that a transmitter and receiver or a transceiver may be used interchangeably in allowing for wireless communication. The physical testing instruments 12 are used by a person performing a physical evaluation upon a patient (not shown) to measure test criteria such as the patient's strength, range of motion, or pressure sensitivity. In performing such a physical evaluation, the data recording device 10 records the measurements taken by the instruments 12. The data recording device 10 may also send signals to the instruments 12 which turn the instruments on or off, or cause the instrument to begin or stop sending measurement data.

In addition to testing instruments, the data recording device may be configured to work in combination with various switches. A foot switch 11 is shown, which may communicate with the data recording device either wirelessly, or through wired communication. The foot switch 11 may perform many functions. For example, the foot switch may be used to record information. An individual who is being tested may step on the foot switch to record the test data for a particular event, such as the maximum force exerted. The test operator may also use the switch, and the foot switch 11 may be used to record data, flag a particular point of interest during a test, start a test, stop a test, or advance to another part of a test.

Also shown is a hand held switch 13. The hand held switch may be used to perform various functions during the test, in a manner similar to the foot switch 11. Accordingly, the hand held switch 13 may have a number of buttons 13b. The buttons may be used to enter data, mark or identify a portion of a test, begin or end a test, move to a new part of a test, etc.

In accordance with one aspect of the invention, multiple instruments 12 can be used in conjunction to form one physical testing system. An example application is dual inclinometry. Dual inclinometry testing is a range of motion testing where two movement sensing devices are used together to determine the range of motion of a specific portion of a subject's body. If a subject is bending his or her neck when tested, the two sensors, which are placed at the base of the subject's neck and top of the head, compensate for movement of the body and give the actual range of motion of the neck. As the subject bends his or her neck, the sensors measure their angular movement from the starting position, and the movement of the sensor at the base of the neck is used to subtract the subject's body movement from the reading of the sensor at the top of the neck, yielding a measurement of the neck movement only.

Current electronic equipment requires that the two sensors for dual inclinometry be connected with a wire. This wire connection can interfere with the ability to conduct tests as it limits the distance between the sensors and may interfere with the patient's mobility. Additionally, the wire can easily become entangled with other wires and objects in the testing facility. Using the technology of the present invention, a radio frequency transceiver and the required electronic circuitry for signal conversion and transmission can be placed in each of the sensors. Each sensor is then in communication with the wireless data recording device 10. The data recording device 10 will cue and receive data from each sensor and make the necessary calculations to determine the range of motion tested. The elimination of the connecting wire between the two sensors allows for greater flexibility in testing; allowing the test subject to perform more complicated tasks while still monitoring their movements. This capability makes possible tests that more closely simulate actual occupational or life activities.

The ability of the data recording device 10 to communicate with multiple testing instruments 12 and display information on a screen 14 enables real time application of test information and more complete test results. Thus, for example, the data recording device 10 can display a movement curve of a patient's limb or other extremity. Not only will it record the overall range of a patient's movement, it can also be used to spot any hesitation or signs of difficulty during the movement, thereby indicating an area of weakness.

According to one aspect of the invention, an adapter 24 can be used to adapt a conventional wired physical testing instrument to function as a wireless physical testing instrument in combination with the present physical testing system. The adapter 24 contains the electronic equipment, such as a wireless transceiver, CPU, and connectors, which are necessary to adapt a wired testing instrument to function with the physical testing system.

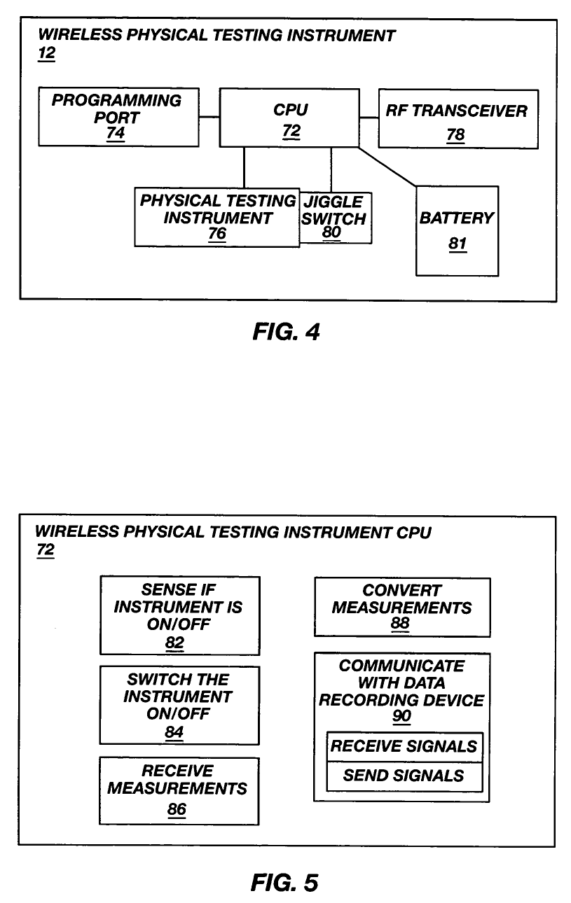

According to another aspect of the present invention, the wireless testing instrument 12 or wireless adapter 24 may contain a switch 80 (FIG. 4) to turn on the instrument. The switch may preferably be a movement sensing switch 80 which is configured to automatically turn an instrument 12 on when picked up or used. When not in use, the testing instrument 12 remains in a standby mode wherein no measurements are taken and signals are not sent to the data recording device 10. When the testing instrument 12 is moved sufficiently, typically because the subject or test operator picks up the instrument 12, the switch 80 generates a signal which turns the instrument on or otherwise commences communication. Many types of switches, such as mechanical or electronic switches, are suitable for use as movement sensing switches 80.

The wireless physical testing system may also contain a display 14. If a display 14 is used, the data recording device 10 would typically send information about the physical evaluation to the display 14. The information shown on the display 14 might include the test measurements, the test duration, information about the test being performed, or patient information.

The physical testing system might also include an input device 16, which may be a keyboard or a mouse, as are common to computers. The input device 16 may be used to create or modify testing procedures, enter information regarding the test, or start or stop the test.

According to another aspect of the invention, the physical testing system may also be configured to communicate with a portable device 18, such as a handheld organizer or laptop computer that is equipped with suitable wireless communication hardware and the software necessary to communicate with and receive data from the testing system. The data recording device 10 may communicate wirelessly or through a wire connection with the portable device 18, or could be integrated or attachable thereto. The portable device 18 may be used during a physical evaluation in a manner similar to the input device 16, sending data to the data recording device 10 to start, stop, or perform the test, and may also receive information from the data recording device 10, such as information about the evaluation being performed.

According to another aspect of the present invention, a physical testing system might include a desired number of physical testing instruments 12 used in combination with a portable device 18 such as a laptop computer or handheld organizer. A portable device 18 which is capable of wireless communication may be configured to communicate directly with the testing instruments 12. If the portable device 18 is not able to communicate directly with the testing instruments 12, a wireless adapter 28 could be used to facilitate communication with the testing instruments 12. The wireless adapter 28 would perform communication functions similar to the data recording device 10. The portable device 18 may be programmed with the software required to communicate with the testing instruments 12, and could perform the same functions that a data recording device 10, display 14, and input device 16 would typically perform. Accordingly, a test operator would only need the portable device 18 and testing instruments 12 to perform physical evaluations. This is a significant advantage where the tests would be performed on location and additional equipment would be cumbersome and difficult to transport to the testing location.

The present invention uses wireless communications devices 20, such as miniaturized radio frequency transmitters and receivers or transceivers or other transmitters and receivers to communicate between the data recording device 10 and the wireless testing instruments 12. In accordance with one aspect of the invention, the testing instruments 12 and data recording device 10 may operate on a single frequency, which may be modulated or communication may be sequenced to facilitate communication between multiple instruments 12 on a single frequency. Additionally, the testing instruments 12 and data recording device 10 may operate in a range of frequencies, hopping between frequencies to facilitate more reliable communication between multiple instruments 12. The software in the data recording device 10 is able to differentiate between different devices, such as the different testing instruments 12 or portable device 18, and is able to identify the testing instruments 12 being used in the physical testing system. The data recording device 10 therefore does not record data signals transmitted by other testing instruments 12 which are not part of the physical testing system. This ability to communicate only with testing instruments 12 and portable devices 18 associated with a particular physical testing system and ignore other testing and wireless devices eliminates ‘cross talking’ between different systems of recording and testing devices and allows multiple wireless physical testing systems to be used in close proximity.

Sequential communication between the data recording device 10 and testing instruments 12 is achieved through dividing the communication time into segments and alternating communication between instruments 12.

According to another aspect of the present invention, the number of testing instruments 12 may be optimized according to the demands of the physical evaluation desired. For example, the use of four instruments 12 is sufficient for most physical evaluations, and a real time display is achieved as compared to where more instruments 12 are used. Of course, more instruments 12 may be used. In practice, the number and type of instruments 12 used may be changed to suit the needs of the particular test being performed. Different number and types of instruments 12 could thus be used to measure the desired aspects of a person's movement and strength while performing a physical evaluation.

In each time segment allocated for communication between the data recording device 10 and a particular testing instrument 12, the data recording device 10 sends a signal requesting data to a particular testing instrument 12. The instrument 12 then sends measurement data back to the data recording device 10. The data recording device 10 then communicates with another testing instrument 12.

For example, if six testing instruments 12 were used for a physical evaluation, the data recording device 10 would cue instrument one and instrument one would send data back to the data recording device 10. The data recording device 10 would then cue instrument two, and so on until the data recording device 10 had received data from all six instruments 12. The data recording device 10 would then start the process over by cueing instrument one for data.

Each time segment for receiving data from an instrument 12 is small, so that the frequency of receiving data is high; many times per second. This provides a set of data for each instrument 12 in which the time between data points is small enough to appear continuous to a test operator and display any important information obtained from the physical evaluation. It is desirable to achieve a real time display of the measurements taken from the testing instruments 12. A real time display of the measurements is achieved when the time delay between the measurement readings from each instrument 12 is sufficiently small so as to be imperceptible to the device operator.

The data recording device 10 may also include wire connections or sockets 22, 26 configured to receive a cable or connector to communicate with the input device 16, the display 14, or possibly the portable device 18. The data recording device 10 may also typically include a data storage device (not shown) and a CPU (not shown). The data storage device records information from the physical evaluation and the CPU processes the information sent to and from the data recording device 10.

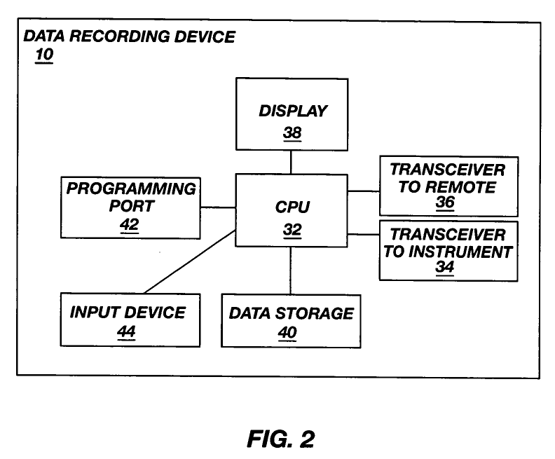

Referring now to FIG. 2, there is shown a schematic block diagram of an embodiment of the data recording device 10. The data recording device 10 utilizes a CPU 32 which interfaces with the other parts of the data recording device 10. There is shown a radio frequency (RF) transceiver 34 to communicate with an instrument 12 (not shown in FIG. 2). One of skill in the art will appreciate that other methods of radio frequency communication, such as using separate transmitters and receivers in place of transceivers, is possible. Through the RF transceiver 34 the data recording device 10 sends signals to physical testing instruments 12 and receives signals from the instruments 12. Another RF transceiver 36 is shown by which the data recording device 10 may communicate to a remote device 18 (not shown in FIG. 2), such as a handheld organizer, laptop computer, or other handheld testing device. It will be appreciated that communication between the data recording device 10 and both the physical testing instruments 12 and the remote device 18 may be accomplished through use of one RF transceiver. Communication with the remote device 18 may also be accomplished through a wire connection (not shown).

There is also shown a display 38 whereby the data recording device 10 displays the readings obtained from the testing instruments 12 in a real time display. The data recording device 10 also comprises a data storage 40 whereby the data recording device 10 stores the data obtained from the testing instruments 12. A programming port 42 is also shown whereby the data recording device 10 is accessible to program or otherwise configure the data recording device 10. The data recording device 10 also includes an input device 44 whereby information regarding the physical evaluation performed may be entered, and whereby commands regarding the evaluation such as starting or stopping a test may be entered. It will be appreciated that the input device 44 may also be used to program or configure the data recording device.

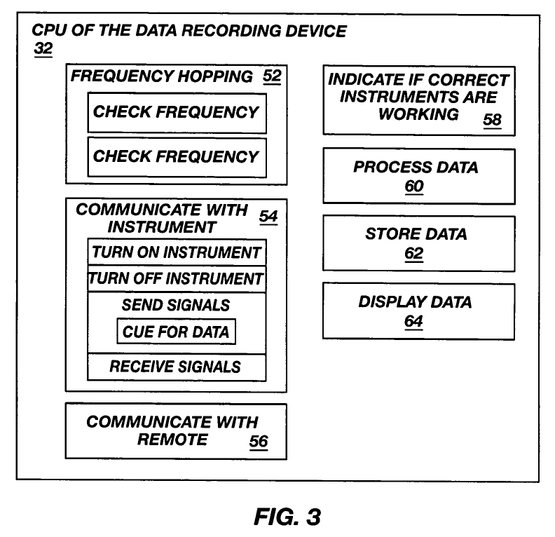

Turning now to FIG. 3, there is shown a schematic block diagram of the functions typically performed by the CPU 32 of the data recording device 10. One function typically performed by the CPU 32 is frequency hopping 52, whereby the CPU 32 transmits on a frequency, and then hops to another frequency, transmits, hops to another frequency, etc. Additionally, the CPU may be used to determine if the signal frequency used is open or if it is noisy or being used by another device. If the frequency is noisy or used the CPU 32 may then change frequency to find an open frequency. The CPU 32 may accordingly be configured to avoid frequencies which are not open. Accordingly, the CPU 32 may send information to the testing instruments 12 to cause the testing instruments 12 to also change frequency to the new frequency used by the data recording device 10. Another important function of the CPU 32 is to communicate 54 with the testing instruments 12. In communicating with the instruments 54, the CPU 32 will both send and receive signals from the instruments 12. The CPU 32 may send a signal to turn an instrument 12 on or off, and sends signals to an instrument 12 to cue the instrument and indicate to the instrument 12 that data should be sent back to the data recording device 10. The CPU 32 may also communicate 56 with a remote device 18. In communicating with a remote device 56, the CPU 32 may send instrument measurement values for display, cue the test operator to take certain actions or perform steps in the test, or confirm that measurements were received properly.

An additional function of the CPU 32 is to determine if the instruments 12 to be used in a test are working properly 58. The CPU 32 may determine if the desired instrument 12 is sending a signal properly, determine if other instruments 12 are sending signals, and alert the test operator of which instruments 12 are working or not working. The CPU 32 also processes data 60. The CPU 32 may convert the data from testing instruments 12 to display the correct reading value and may associate the data to the specific body part being tested. The CPU 32 also may store data 62 by sending it to the data storage 40. Additionally, the CPU 32 displays data 64 by sending data to the display 38. A real time display is provided whereby the test operator may view the test results on the display 38 as they are generated by the physical testing instruments 12.

Referring now to FIG. 4, a schematic block diagram of a wireless physical testing instrument 12 is shown. The instrument 12 contains a CPU 72. The CPU 72 is connected to a programming port 74 whereby the CPU 72 is configured to communicate with the testing instrument 76 and the data recording device 10. The instrument 12 also contains a RF transceiver 78 to send signals to and receive signals from the data recording device 10. The instrument 12 also may contain a jiggle switch 80, whereby movement of the instrument causes the switch to send a signal to the CPU 72, and the instrument 12 is switched from a standby mode to a powered on mode. The instrument will also typically contain a battery 81.

In the situation where the jiggle switch 80 is a mechanical switch, a resilient switch which may be mounted inside the instrument 12 is bent temporarily by the movement. When bent, the resilient member of the switch contacts a stationary member of the switch and causes electrical contact between the resilient member and the stationary member. The resilient member then returns to its unbent position. The momentary electrical contact between the switch members signals to the instrument CPU 72 that the instrument 12 is in use and electrical power is sent to the instrument 12. The instrument 12 then remains on for a predetermined period of time. Other types of mechanical switches, such as a roller ball switch, could be used. Additionally, other types of switches such as digital accelerometers are available and could be used.

According to another aspect of the present invention, the wireless instrument 12 may be switched on by a radio frequency signal from the data recording device 10. The instrument 12 would remain in a standby mode until turned on or otherwise actuated. The instrument 12 may also begin taking measurements upon receiving a signal from the data recording device 10. The instrument 12 then begins taking measurements and sending information to the data recording device 10. In a similar fashion, at the end of the test the data recording device 10 can send a signal to the instrument 12 which causes the instrument 12 to return to a standby mode. Thus, in performing a physical evaluation, the data recording device 10 could be used to control the testing instruments 12.

Referring to FIG. 5, a schematic block diagram of the functions performed by the instrument CPU 72 is shown. The CPU 72 may sense if the instrument 12 is on or off 82, and switch the instrument 12 on or off 84 when a signal is received from the jiggle switch 80 or the data recording device 10. Additionally, the CPU 72 receives measurements 86 from the instrument 12. In receiving measurement data 86 the CPU 72 typically converts 88 the measurement data to the proper measurement value. The CPU 72 also communicates 90 with the data recording device 10.

In communicating, the CPU 72 may need to change the frequency used for communication. The data recording device 10 may use frequency hopping, whereby the data recording device 10 monitors the communication frequency and changes to another frequency if the current communication frequency is noisy or used by another device. Additionally, frequency hopping may involve changing frequencies regularly, regardless of whether a frequency is being used or is noisy. The communication may be accomplished by hopping frequencies at regular intervals and or by using a predetermined list of frequencies. Accordingly, the testing instruments 12 may receive signals from the data recording device 10 indicating that a new frequency is used for communication. The testing instrument CPU 72 would then change the frequency used by the testing instrument 12 for communication. Communication 90 also comprises receiving and sending signals from the data recording device 10. During the course of a physical evaluation, the data recording device 10 might send a signal to the testing instrument 12 indicating that a measurement reading should be sent to the data recording device 10 and the instrument 12 would then send that measurement.

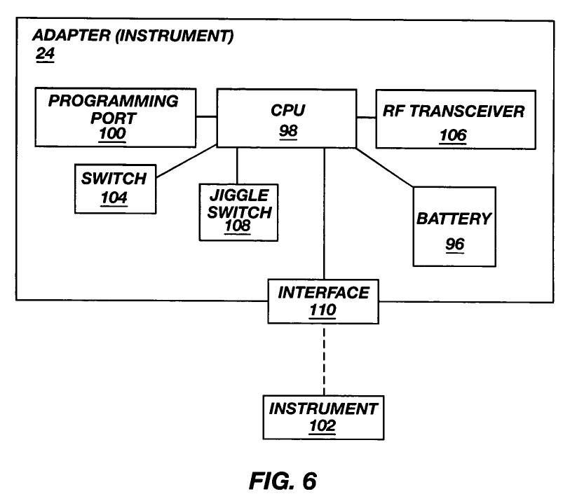

Turning now to FIG. 6 a schematic block diagram of an adapter 24 to adapt a wired physical testing instrument 102 to function as a wireless physical testing instrument is shown. According to one aspect of the present invention, a wireless adapter 24 can be made to be attachable to an existing wired electronic testing instrument 102 to convert the instrument to wireless use and function with the present wireless physical testing system.

The adapter 24 utilizes a wireless transceiver 106 to communicate with the data recording device 10 in the same manner as a wireless testing instrument 12 as disclosed by the present invention. The adapter 24 also contains internal circuitry 98 to convert the signal generated by the instrument 102 into an appropriate signal that is understood by the data recording device 10. The adapter 24 also uses a suitable means 104 for selecting the type of testing instrument 102 adapted to wireless use so that the correct measurement value is sent to the data recording device 10. By way of example, the switch means 104 could comprise a switch or switches, a dial, or buttons for selecting the brand or type of instrument 102 adapted. The switch 104 may also be a small dip type switch containing a number of switches whereby many different combinations of switch positions are possible, allowing for selection of many different types or brands of testing instruments 102. Instead of a switch, the adapter may simply be programmed for use with a particular instrument or type of instrument.

One of skill in the art will appreciate that many means 104 are available for selecting the type of instrument 102 to be adapted for wireless use and the present invention is not intended to be limited by the means listed. The adapter 24 may also include a battery 96 to supply power to the instrument 102 adapted to wireless use. The adapter 24 also utilizes an appropriate housing to contain the necessary parts and attach the adapter 24 to the instrument 102, as well as an electrical connector or interface 110 to facilitate communication between the electronic instrument 102 and wireless adapter 24. Multiple styles of connector 110 may be used to accommodate different brands or types of testing instruments 102. Additionally, different shapes and configurations of housings may be used if necessary. As such, the adapter 24 is configured to adapt most brands and styles of wired electronic physical testing instruments 102 to wireless use. It is contemplated that a single adapter 24 could retrofit many, if not all, brands and types of wired electronic physical testing instruments 102.

The adapter 24 includes a CPU 98 which is connected to a programming port 100 for configuring the CPU 98 to communicate with a wired testing instrument 102. The adapter 24 further includes a RF transceiver 106 whereby communication with the data recording device 10 is accomplished. The adapter 24 also may include a jiggle switch 108 which senses movement of the adapter 24 and instrument 102 and causes the CPU 98 to switch the instrument 102 on from a standby state.

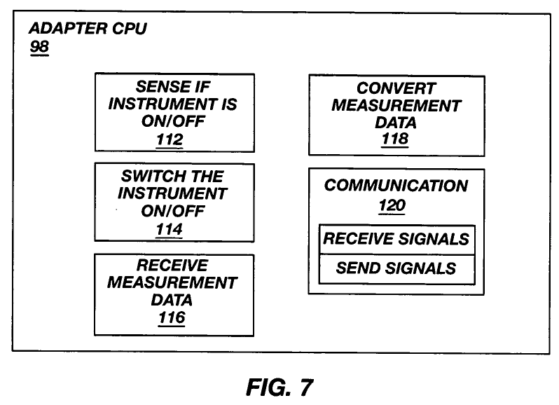

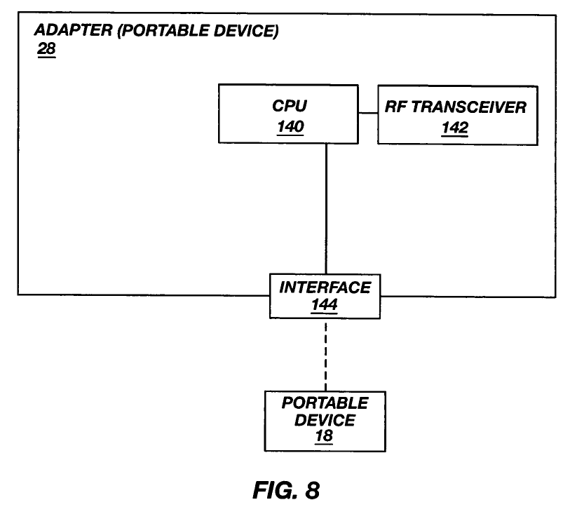

Turning now to FIG. 7 a schematic block diagram of the functions performed by the CPU 98 of the adapter 24 is shown. It is intended that the CPU 98 may sense if the instrument 102 is on or off 112, and also may switch the instrument 102 on or off 114 when a signal is received from the jiggle switch 108 or the data recording device 10. The CPU 98 receives data 116 from the instrument 102 and converts 118 that data to the proper measurement value. Converting measurement data 118 typically would include the CPU 98 knowing what type and brand of instrument 102 is connected to the adapter 24 and then converting the measurement data to a measurement value. The CPU 98 may determine what type and brand of instrument 102 is connected by the switch 104 settings or by information entered through the programming port 100. The CPU 98 then communicates 120 with the data recording device 10. Communication 120 typically involves receiving a signal from the data recording device 10 indicating that data should be sent to the data recording device 10 and sending that data.

Turning now to FIG. 8, a schematic diagram is shown of an adapter 28 configured to adapt a portable device 18 to wireless use in a wireless physical testing system. It is desireable in many situations to adapt a portable device 18, such as a hand held organizer, to communicate with testing instruments 12. This allows great flexibility in performing physical evaluations on location while transporting as little equipment as possible. The adapter 28 may typically contain a CPU 140 to process the signals sent and received by the portable device 18. The CPU 140 is connected to a wireless transceiver 142 through which the adapter 28 sends and receives signals. The adapter 28 also contains an interface 144 by which the adapter 28 is connected to the portable device 18. The interface 144 also transfers signals between the adapter 28 and the portable device 18.

In operation, a test operator would connect the adapter 28 to the portable device 18. The portable device 18 typically would have the software necessary to transfer information to and from the adapter 28, as well as software which controls the physical evaluation. The software would typically allow the test operator to collect, display, and store the information from the testing instruments 12. During a test, the portable device 18 would perform functions similar to the data recording device 10 by controlling the testing instruments 12, managing the communication frequency, and managing the data produced by the testing instruments 12. Thus, the portable device 18 performs similar functions as the CPU 32 of the data recording device 10.

The portable device 18 may check the communication frequency for noise or interference and possibly change frequency, communicate with the testing instruments, indicate if the correct instruments are working, process data, store data, display data, and control the testing instruments 12. The portable device 18 may be configured to check frequencies for noise and operate on a clean frequency, or may be configured to simply operate on a predetermined frequency or change frequencies according to a predetermined schedule. When signals are sent from the portable device 18, the interface 144 transfers the signals to the CPU 140 of the adapter 28. The CPU 140 processes the signals as required for communication, and sends the signals to the RF transceiver 142. The RF transceiver 142 sends the signals to the testing instruments 12. Similarly, when a testing instrument 12 sends a signal to the portable device 18, the signal is received by the RF transceiver 142 and sent to the CPU 140. The CPU 140 then processes the signal as may be necessary prior to sending the signal to the portable device 18, through the interface 144.

There is thus disclosed an improvement in a wireless physical testing system. The wireless physical testing system allows for much greater accuracy and flexibility in performing physical evaluations. The wireless physical testing system also allows a test operator to design and implement physical tests which accurately simulate life activities. One of skill in the art will appreciate that numerous modifications may be made to the present invention without departing from the scope of the invention. The appended claims are intended to cover such modifications.

What is claimed is:

1. A wireless physical testing system for testing physical abilities of a patient comprising:
a plurality of electronic testing devices; and
a data recording device configured for wireless communication with the plurality of testing devices;
and wherein the wireless physical testing system simultaneously measures at least two of the group consisting of range of motion, flexibility, movement speed, strength, pain sensitivity, heart rate, and body temperature.

2. The wireless physical testing system according to claim 1 wherein the data recording device further comprises a handheld organizer.

3. The wireless physical testing system according to claim 1 wherein the data recording device further comprises a laptop computer.

4. The wireless physical testing system according to claim 1 wherein the data recording device communicates to at least two testing devices simultaneously.

5. The wireless physical testing system according to claim 4 wherein simultaneous communication is achieved by rapid sequential communication with the testing devices.

6. The wireless physical testing system according to claim 1 wherein the data recording device displays the data from the testing devices in a real time display.

7. The wireless physical testing system according to claim 1 wherein the data recording device communicates to multiple testing devices sequentially.

8. The wireless physical testing system according to claim 1, wherein the testing devices comprise at least 2 of the group consisting of algometers, inclinometers, dual inclinometers, goniometers, strength testing instruments, foot switches, and hand switches.

9. A wireless physical testing system comprising:
a plurality of physical testing devices configured for testing range of motion, strength, speed, flexibility, or pain tolerance;
a data recording device capable of transmitting and receiving data from the plurality testing devices; and
wireless communication between the testing devices and the recording device.

10. The wireless physical testing system according to claim 9, wherein multiple testing devices are used simultaneously for a single test.

11. The wireless physical testing system according to claim 9, wherein the recording device communicates to multiple testing devices sequentially.

12. The wireless physical testing system according to claim 9, wherein the recording device identifies the signals from the testing devices used in the physical test performed and rejects signals from other testing instruments.

13. The wireless physical testing system according to claim 9, wherein the testing devices turn on automatically when moved.

14. The wireless physical testing system according to claim 9, wherein the testing device turns on automatically when a signal is sent to the testing device from the recording device.

15. The wireless physical testing system according to claim 9, further comprising a real time display of the measurements obtained from the testing devices.

16. A physical testing instrument for dual inclinometry comprising two sensors for determining relative positions of body parts wherein the two sensors are not connected to another sensor or recording device by wires and wherein the sensors communicate with a recording device through wireless communication.

17. An adapter for adapting wired electronic physical testing devices to communicate with a data recording device through wireless communication comprising an interface for communicating with the testing device and a transmitter for conveying signals to a data recording device.

18. The adapter according to claim 17, wherein the adapter is configured to work with multiple brands and models of physical testing devices.

19. The adapter according to claim 18, further comprising a switch for selecting what model and brand of testing device is to be adapted to wireless use.

20. The adapter according to claim 17, further comprising a power source for providing power to the physical testing device.

21. A physical testing system having the adapter according to claim 17 and a data recording device, wherein the data recording device communicates simultaneously with multiple adapters.

22. The system according to claim 21, wherein the data recording device communicates sequentially with multiple adapters.

23. A method for physical testing which comprises:
requiring a subject to perform occupational or life activities; and
monitoring the subject with at least two wireless physical testing devices from the group consisting of inclinometers, dual inclinometers, algometers, goniometers, and strength testing devices.

24. The method according to claim 23, wherein the physical testing devices are used simultaneously.

25. The method according to claim 23, wherein the method further comprises recording the measurements from the multiple wireless physical testing devices with a single data recording device.

26. The method according to claim 23, wherein the recording device communicates with the testing devices sequentially.

27. The method according to claim 23, wherein the recording device communicates with the testing devices simultaneously.

28. The method according to claim 23, wherein the recording device automatically recognizes and accepts the signals from the testing devices used in the physical test and rejects signals from other wireless testing devices.

29. A method for physical testing which comprises:
selecting a wireless data recording device which communicates with a plurality of wireless testing devices;
selecting at least two of the plurality wireless testing devices;
having a patient perform an occupational or lifestyle activity which involves simultaneous use of the at least two wireless testing devices;
measuring the patient's performance with the at least two wireless testing devices; and
recording the measurements taken by the at least two wireless testing devices with the data recording device.

30. The method according to claim 29, wherein the wireless testing devices are selected from the group consisting of inclinometers, dual inclinometers, algometers, goniometers, strength testing devices, heart rate monitors, and body temperature monitors.

31. The method according to claim 29, wherein the data recording device communicates with multiple testing devices through sequential communication.

32. The method according to claim 29, wherein the method further comprises displaying the test measurements in a real time display.

33. The method according to claim 29, wherein the method further comprises selecting a data recording device which automatically detects and accepts the signals from the testing devices used in the physical test and which does not accept signals from other wireless testing devices.

34. A method for adapting electronic physical testing devices for use with a wireless testing system which comprises:
selecting an electronic testing device;
fitting the device with an adapter capable of communication with the electronic testing device and capable of wireless communication with a wireless receiver; and
selecting a wireless receiver.

35. The method according to claim 34, wherein the method further comprises selecting a wireless receiver that is capable of communication with multiple testing devices at the same time.

36. The method according to claim 34, wherein the method further comprises selecting an adapter that is capable of communication with multiple brands and models of electronic physical testing devices.

37. The method according to claim 36, wherein the method further comprises selecting an adapter with a switch to select the model and brand of electronic testing device adapted to wireless use and positioning the switch to enable the adapter to communicate with the desired electronic physical testing device.

38. The method according to claim 34, wherein the method further comprises selecting an adapter which supplies power to the electronic testing device adapted to wireless use.

39. A method for physical testing which comprises using multiple wireless testing devices to simultaneously measure at least two of the group consisting of strength, range of motion, pain sensitivity, and pressure sensitivity while the test subject performs an occupational, lifestyle, or diagnostic activity.

40. The method for physical testing according to claim 39, wherein the method further comprises selecting multiple wireless testing devices which communicate with a single data recording device.
